(19) United States
(12) Patent Application Publication
(10) Pub. No.: US 2023/0085128 A1
(43) Pub. Date: Mar. 16, 2023

(54) INJECTION DEVICE FOR SUBCUTANEOUS SEQUENTIAL DELIVERY OF GAS AND A DRUG

(71) Applicant: WEST PHARMACEUTICAL SERVICES, INC., Exton, PA (US)

(72) Inventors: Brian COSTELLO, Whitehouse Station, NJ (US); Alex LYNESS, West Chester, PA (US); Raymond PROTASIEWICZ, Whippany, NJ (US); Geoffrey T. ROUIN, Morristown, NJ (US)

(21) Appl. No.: 17/794,677

(22) PCT Filed: Dec. 22, 2020
(86) PCT No.: PCT/US2020/066505

§ 371 Date: Jul. 22, 2022

Related U.S. Application Data
(60) Provisional application No. 62/965,631, filed on Jan. 24, 2020.

(51) Int. Cl. A61M 5/14; A61M 5/168

(52) U.S. Cl. CPC A61M 5/1408; A61M 5/16827; A61M 2005/006

(57) ABSTRACT

A parenteral injection device comprises a housing defining an internal cavity and an output, a first reservoir disposed within the internal cavity, where the first reservoir defines a first chamber that is configured to store a drug and is in fluid communication with the output, a first drive mechanism to selectively force the drug from the first chamber, and a second reservoir disposed within the internal cavity, wherein the second reservoir defines a second chamber that is configured to store a gas and is in fluid communication with the output. The injection device further comprises a second drive mechanism to selectively force the gas from the second chamber, and a controller in signal communication with the first and second drive mechanisms, such that the controller is configured to selectively direct the first and second drive mechanisms to force the drug and gas from the first and second chambers, respectively, to the output in a predetermined sequence.

### CROSS-REFERENCE TO RELATED APPLICATIONS

This application claims the benefit of U.S. Provisional Patent App. No. 62/965,631, filed Jan. 24, 2020, the entire contents of which are hereby incorporated by reference as if set forth in its entirety herein.

### TECHNICAL FIELD

This application is directed to an injection device. Specifically, this application relates to an injection device for sequentially providing a drug and a gas to a patient.

### BACKGROUND

Hypodermic injections allow a variety of therapies to be administered in subcutaneous, intramuscular, or intravenous manners. Many therapeutic agents, specifically biologics and other advanced therapies, must be administered through these routes to maintain their structural integrity, and thus their proper biological functionality. However, subcutaneous injections are typically associated with patient discomfort, which can result from the pH of the drug, size of the drug dose, or sensitivity of the patient. In response, healthcare professionals have begun introducing air pockets along with drug doses, which may reduce injections site reactions, such as bruising and pain.

Currently, healthcare professionals must perform this operation manually. For example, healthcare professionals can draw an amount of air between a syringe plunger and a drug to be injected, as well as replacing the needle after the drug has been drawn into the syringe, thus priming the new needle with air. This can be referred to colloquially as preparing a “sandwich shot” of a drug and gas. However, such a process requires a trained healthcare professional to perform time-consuming actions, and thus such a process must occur at a healthcare facility, which increases time and cost associated with an injection for both healthcare providers and patients. This manual approach is also highly variable with regards to the volumes of liquid and gas and their ratio, meaning that replicating the exact dosing regime is difficult and that inconsistency may affect the patient experience of uptake of the medication. Further, air injected into a patient through this process may be unfiltered, which potentially risks introducing environmental contaminants into a patient.

As a result, there is a need for a device that automatically performs a sequential drug and filtered gas injection operation in an automated and controlled manner without the aid of a healthcare professional.

### SUMMARY

An embodiment of the present disclosure is an injection device for sequentially and parenterally providing a drug and a gas to a patient, where the injection device comprises a housing defining an internal cavity and an output, and a first reservoir disposed within the internal cavity, where the first reservoir defines a first chamber that is configured to store a drug and is in fluid communication with the output. The injection device also comprises a first drive mechanism operably attached to the first reservoir so as to selectively force the drug from the first chamber, and a second reservoir disposed within the internal cavity, wherein the second reservoir defines a second chamber that is configured to store a gas and is in fluid communication with the output. The injection device further comprises a second drive mechanism operably attached to the second reservoir so as to selectively force the gas from the second chamber, and a controller in signal communication with the first and second drive mechanisms, such that the controller is configured to selectively and independently direct the first and second drive mechanisms to force the drug and gas from the first and second chambers, respectively, to the output in a predetermined sequence.

Another embodiment of the present disclosure is an injection device for sequentially and parenterally providing a drug and a gas to a patient, where the injection device comprises a housing defining an internal cavity, an input, and an output, a first drive mechanism in fluid communication with the input, where the first drive mechanism is configured to selectively draw gas through the input and force the gas to the output, and a first reservoir disposed within the internal cavity, where the first reservoir defines a first chamber that is configured to store a drug and is in fluid communication with the output. The injection device also comprises a second drive mechanism operably attached to the first reservoir so as to selectively force the drug from the first chamber, and a controller in signal communication with the first and second drive mechanisms, such that the controller is configured to selectively and independently direct the first and second drive mechanisms to force the gas and drug from the input and first chamber, respectively, to the output in a predetermined sequence.

### BRIEF DESCRIPTION OF THE DRAWINGS

The foregoing summary, as well as the following detailed description, will be better understood when read in conjunction with the appended drawings. The drawings show illustrative embodiments of the disclosure. It should be understood, however, that the application is not limited to the precise arrangements and instrumentalities shown.

FIG. 1A is a schematic view of an injection device according to an embodiment of the present disclosure;

FIG. 1B is a schematic view of an injection device according to another embodiment of the present disclosure;

FIG. 2A is a schematic view of an injection device according to an alternative embodiment of the present disclosure;

FIG. 2B is a schematic view of an injection device according to a further embodiment of the present disclosure;

FIG. 3A is a process flow diagram illustrating a method of sequentially injecting a drug and a gas according to an embodiment of the present disclosure;

FIG. 3B is a further process flow diagram illustrating a method of sequentially injecting a drug and a gas according to an embodiment of the present disclosure;

FIG. 3C is an additional process flow diagram illustrating a method of sequentially injecting a drug and a gas according to an embodiment of the present disclosure;

FIG. 3D is a further process flow diagram illustrating a method of sequentially injecting a drug and a gas according to an embodiment of the present disclosure;

FIG. 4A is a process flow diagram illustrating a method of sequentially injecting a first drug, a second drug, and a gas according to an embodiment of the present disclosure;

FIG. 4B is a further process flow diagram illustrating a method of sequentially injecting a first drug, a second drug, and a gas according to an embodiment of the present disclosure;

FIG. 4C is an additional process flow diagram illustrating a method of sequentially injecting a first drug, a second drug, and a gas according to an embodiment of the present disclosure;

FIG. 4D is a process flow diagram illustrating a method of sequentially injecting a first drug, a second drug, and a gas according to an embodiment of the present disclosure; and

FIG. 4E is a further process flow diagram illustrating a method of sequentially injecting a first drug, a second drug, and a gas according to an embodiment of the present disclosure.

### DETAILED DESCRIPTION OF ILLUSTRATIVE EMBODIMENTS

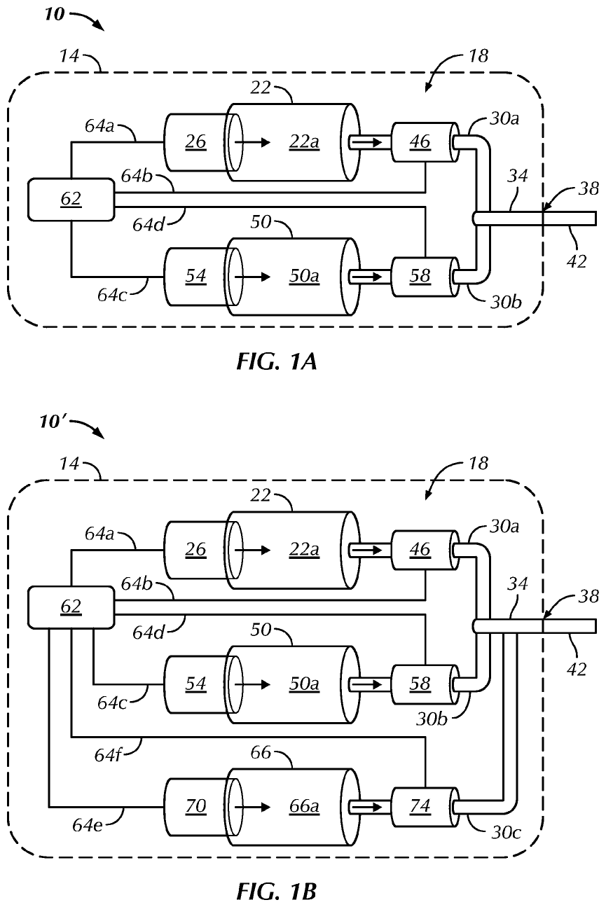

Referring to FIG. 1A, an embodiment of the present disclosure comprises an injection device 10 configured to store and sequentially provide a drug and a gas to a patient. The injection device 10 can have a housing 14 that is substantially hollow and is comprised of a metal or plastic. In one embodiment, the housing 14 can include an adhesive directly or indirectly attached thereto such that the injection device 10 is a wearable device. However, it is contemplated that the injection device 10 can comprise a device other than a wearable device. For example, the injection device can comprise an autoinjector, an infusion pump, or an injector configured to deliver a substance to a patient through a catheter. However, other devices capable of injecting a substance are contemplated.

The housing 14 can define a cavity 18 therein, where components of the injection device 10 are configured to reside within the cavity 18. The housing 14 also can define an output 38, which will be described further below. The injection device 10 can include a first reservoir 22 disposed within the internal cavity 18, where the first reservoir 22 defines a first chamber 22a. The first reservoir 22 can be configured as a plastic or glass syringe or cartridge configured to be inserted into the injection device 10 upon preparation or assembly of the injection device. Such a reservoir can be removably disposed within the cavity 18, such that a user can remove the first reservoir 22 upon depletion of its contents and replace the first reservoir 22. However, it is contemplated that the first reservoir 22 may be irremovably disposed within the cavity 18. In another embodiment, it is contemplated that the first reservoir 22 can define a receiving space incorporated into the design of the injection device 10 and is configured to be filled from an external source.

The first chamber 22a of the first reservoir 22 is configured to store a drug. Additionally, the first chamber 22a is in fluid communication with the output 38. To force the drug from the first chamber 22a to the output 38, the injection device 10 can include a first drive mechanism 26 operably attached to the first reservoir 22. The first drive mechanism 26 can be configured to selectively force the drug from the first chamber 22a, as will be described below. In one embodiment, the first drive mechanism 26 can comprise a motor operably connected to a telescopic screw assembly (TSA), where the TSA is comprised of a plurality of concentrically-arranged screw segments configured to axially advance a plunger (not shown) through the first chamber 22a so as to force the drug from the first reservoir 22. However, it is also contemplated that the first drive mechanism 26 can include an electromechanical pump, piezoelectric actuator, shape memory alloy, peristaltic pump, spring, electro-chemical drive mechanism, and/or any other suitable device for dispensing the drug.

As stated above, the first drive mechanism 26 can be selectively actuated so as to force the drug from the first reservoir 22. To accomplish this, the first drive mechanism 26 can be in signal communication with a controller 62 through a signal connection 64a. The signal connection 64a can comprise a wired and/or a wireless connection. The controller 62 can comprise any suitable computing device configured to provide a software application for monitoring and controlling various operations of the injection device as described herein. For example, the controller 62 can include a processor, a memory, and/or a human-machine interface (HMI) device. The memory can be volatile (e.g., some types of RAM, etc.), non-volatile (e.g., ROM, flash memory, etc.), or a combination thereof. Further, the controller 62 can include additional storage devices, such as removable storage devices and/or non-removable storage devices. Exemplary HMI devices can comprise buttons, soft keys, speakers, display screens, lights, microphones, etc. As such, the controller 62 can be directly controlled by a user of the injection device 10. Alternatively, the controller 62 can include automated operation instructions such that the controller 62 can automatically control the injection device 10 according to a predetermined operation automatically or upon instruction from the user. Further, the controller 62 can be wirelessly controlled by a remote device (not shown), such as through Bluetooth, WIFI, near-field communication (NFC), etc., as well as be configured to wirelessly transmit and/or receive operational information to and/or from such a remote device.

Continuing with FIG. 1A, the first drive mechanism 26 is configured to selectively force the drug from the first chamber 22a. The injection device 10 can include a first fluid path 30a that extends from the first chamber 22a to an outlet path 34. The first fluid path 30a and the outlet path 34 can comprise any conventional fluid path capable of receiving and directing a flow of a drug. For example, the first fluid path 30a and/or the outlet path 34 can be a rigid or flexible tube comprised of metal, plastic, rubber, etc. The outlet path 34 can extend from the first fluid path 30a to an output 38 defined by the housing 14 of the injection device 10. At the output 38, the outlet path 34 can be in fluid communication with a cannula 42 extending from and/or at least partially exterior to the housing 14. Though depicted as exterior to and extending from the housing 14, it is contemplated that the cannula 42 is movable relative to the housing 14. For example, the cannula 42 can be moveable from a retracted position in which the cannula 42 is positioned at least partially within the housing 14 to an extended position in which the cannula 42 at least partially extends from the housing 14. In one embodiment, the cannula 42 comprises a hollow needle with a sharp tip for piercing the skin of a user and subcutaneously delivering a substance, where the needle can be linear or have angularly extending portions. However, other cannula embodiments are contemplated. Additionally, it is contemplated that the cannula 42 can be spaced a distance from the housing 14, such as when the injection device 10 comprises an infusion pump.

To further control the flow of the drug from the first chamber 22a to the outlet path 34, a first valve 46 can be fluidly positioned between the first chamber 22a and the output 38. Specifically, the first valve 46 can be incorporated into the first fluid path 30a. In operation, the first valve 46 is configured to selectively inhibit the flow of the drug from the first chamber 22a to the output 38. To accomplish this, the first valve 46 can be in signal communication with the controller 62 through signal connection 64b, which can be a wired and/or wireless connection. The controller 62 can thus instruct the 46 to selectively block or allow flow of the drug to the output 38 based upon a predetermined dispensing sequence, as will be described further below. The first valve 46 can be a solenoid valve, electrostatic valve, piezoelectric valve, shape-memory actuated valve, coaxial valve, or an angle seat valve. However, the present disclosure is not meant to be limited to such and other types of valves are contemplated.

With continued reference to FIG. 1A, the injection device 10 can further include a second reservoir 50 disposed within the internal cavity 18, where the second reservoir 50 defines a second chamber 50a. Like the first reservoir 22, the second reservoir 50 can be configured as a plastic or glass syringe or cartridge configured to be inserted into the injection device 10 upon preparation or assembly of the injection device 10. Such a reservoir can be removably disposed within the cavity 18, such that a user can remove the second reservoir 50 upon depletion of its contents and replace the second reservoir 50. However, it is contemplated that the second reservoir 50 may be irremovably disposed within the cavity 18. In another embodiment, it is contemplated that the second reservoir 50 can define a receiving space incorporated into the design of the injection device 10 and is configured to be filled from an external source.

Unlike the first chamber 22a of the first reservoir 22, the second chamber 50a of the second reservoir 50 is configured to store a gas. In one embodiment, the gas itself is a drug having a therapeutic effect, though other types of gasses are contemplated. The gas can be introduced to a patient through the cannula 42 before or after the drug so as to reduce discomfort during an injection, as will be described further below. Additionally, the second chamber 50a is in fluid communication with the output 38. To force the gas from the second chamber 50a to the output 38, the injection device 10 can include a second drive mechanism 54 operably attached to the second reservoir 50. The second drive mechanism 54 can be configured to selectively force the gas from the second chamber 50a, as will be described below. In one embodiment, the second drive mechanism 54 can comprise a motor operably connected to a TSA, where the TSA is configured to axially advance a plunger (not shown) through the second chamber 50a so as to force the gas from the second reservoir 50. However, it is also contemplated that the second drive mechanism 54 can include an electromechanical pump, piezoelectric actuator, shape memory alloy, peristaltic pump, spring, electro-chemical drive mechanism, and/or any other suitable device for causing axial actuation of a plunger.

As stated above, the second drive mechanism 54 can be selectively actuated so as to force the gas from the second reservoir 50. To accomplish this, the second drive mechanism 54 can be in signal communication with a controller 62 through a signal connection 64c. The signal connection 64c can comprise a wired and/or a wireless connection. The second drive mechanism 54 is configured to selectively force the drug from the second chamber 50a. The injection device 10 can include a second fluid path 30b that extends from the second chamber 50a to the outlet path 34. The second fluid path 30b, like the first fluid path 30a and the outlet path 34, can comprise any conventional fluid path capable of receiving and directing a flow of a gas. For example, the second fluid path 30b can be a rigid or flexible tube comprised of metal, plastic, rubber, etc.

To further control the flow of the drug from the second chamber 50a to the outlet path 34, a second valve 58 can be fluidly positioned between the second chamber 50a and the output 38. Specifically, the second valve 58 can be incorporated into the second fluid path 30a. In operation, the second valve 58 is configured to selectively inhibit the flow of the drug from the second chamber 50a to the output 38. To accomplish this, the second valve 58 can be in signal communication with the controller 62 through signal connection 64d, which can be a wired and/or wireless connection. The controller 62 can thus instruct the second valve 58 to selectively block or allow flow of the gas to the output 38 based upon a predetermined dispensing sequence, as will be described further below. The second valve 58 can be a solenoid valve, electrostatic valve, piezoelectric valve, shape-memory actuated valve, coaxial valve, or an angle seat valve. However, the present disclosure is not meant to be limited to such and other types of valves are contemplated.

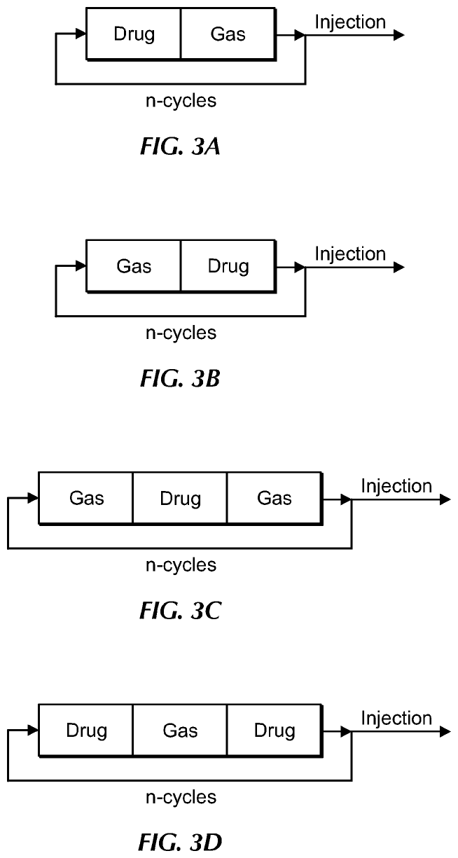

As described above, the controller 62 is in signal communication with the first and second drive mechanisms 26, 54 via signal connections 64a, 64c, respectively. As a result, the controller 62 can selectively and individually direct the first and second drive mechanisms 26, 54 to force the drug and gas from the first and second chambers 22a, 50a, respectively, to the output 38 in a predetermined sequence. The predetermined sequence can be stored in a memory of the controller 62, can be manually input by a user, or can be provided to the controller 62 wirelessly from a remote device. Similarly, the controller 62 is in signal communication with the first and second valves 46, 58 via signal connections 64b, 64d, respectively. As a result, the controller 62 can selectively and individually control actuation of each of the first and second valves 46, 68. One embodiment of the predetermined sequence can comprise a discrete amount of the gas followed by a discrete amount of the drug. However, potential embodiments of the predetermined sequences that the injection device 10 is capable of injecting are described further below with respect to FIGS. 3A-3D.

Now referring to FIG. 1B, another embodiment of an injection device 10′ is shown. The injection device 10′ has similar components to the injection device 10. As a result, like reference numbers will be utilized in connection with the injection device 10′, and descriptions of similar components will not be repeated for brevity. As shown in FIG. 1B, the injection device 10′ can include a third reservoir 66 disposed within the internal cavity 18, where the third reservoir 66 defines a second chamber 66a. Like the first and second reservoirs 22, 50, the third reservoir 66 can be configured as a plastic or glass syringe or cartridge configured to be inserted into the injection device 10′ upon preparation or assembly of the injection device. Such a reservoir can be removably disposed within the cavity 18, such that a user can remove the third reservoir 66 upon depletion of its contents and replace the third reservoir 66. However, it is contemplated that the third reservoir 66 may be irremovably disposed within the cavity 18. In another embodiment, it is contemplated that the third reservoir 66 can define a receiving space incorporated into the design of the injection device 10 and is configured to be filled from an external source.

The third chamber 66a of the third reservoir 66 is configured to store a second drug or liquid substance (buffer, excipient, etc.) that can enhance the effect or comfort of the first drug and/or injection of gas, where the drug stored by the first reservoir 22 can be considered a first drug. It is contemplated that the first and second drugs can be the same, such that the third reservoir 66 represents an additional supply of the same drug contained within the first reservoir 22. However, it is further contemplated that the first and second drugs can be different. For example, the first and second drugs can comprise drugs that are injected into a patient during the same injection process but cannot be mixed prior to injection to maintain stability of one or more of the drugs or the efficacy of a therapy is improved via injection of a second drug or liquid substance. The third chamber 66a is in fluid communication with the output 38. To force the second drug from the third chamber 66a to the output 38, the injection device 10′ can include a third drive mechanism 70 operably attached to the third reservoir 66. The third drive mechanism 70 can be configured to selectively force the second drug from the third chamber 66a as will be described below. In one embodiment, the third drive mechanism 70 can comprise a TSA configured to axially advance a plunger (not shown) through the third chamber 66a so as to force the second drug from the third reservoir 66. However, it is also contemplated that the third drive mechanism 70 can include an electromechanical pump, piezoelectric actuator, shape memory alloy, peristaltic pump, spring, electro-chemical drive mechanism, and/or any other suitable device for causing axial actuation of a plunger.

As stated above, the third drive mechanism 70 can be selectively actuated so as to force the second drug from the third reservoir 66. To accomplish this, the third drive mechanism 70 can be in signal communication with the controller 62 through a signal connection 64e. The signal connection 64e can comprise a wired and/or a wireless connection. The injection device 10′ can include a third fluid path 30c that extends from the third chamber 66a to the outlet path 34. The third fluid path 30c, like the first and second fluid paths 30a, 30b and the outlet path 34, can comprise any conventional fluid path capable of receiving and directing a flow of a drug or liquid substance. For example, the third fluid path 30c can be a rigid or flexible tube comprised of metal, plastic, rubber, etc. The configuration of first, second, and third fluid paths 30a-30c shown in FIG. 1B is illustrative and different combinations and embodiments of fluid communication are envisaged.

To further control the flow of the drug from the third chamber 66a to the outlet path 34, a third valve 74 can be fluidly positioned between the third chamber 66a and the output 38. Specifically, the third valve 74 can be incorporated into the third fluid path 30c. In operation, the third valve 74 is configured to selectively inhibit the flow of the drug from the third chamber 66a to the output 38. To accomplish this, the third valve 74 can be in signal communication with the controller 62 through signal connection 64f, which can be a wired and/or wireless connection. The controller 62 can thus instruct the third valve 74 to selectively block or allow flow of the second drug or liquid substance to the output 38 based upon a predetermined dispensing sequence, as will be described further below. The third valve 74 can be a solenoid valve, electrostatic valve, piezoelectric valve, shape-memory actuated valve, coaxial valve, or an angle seat valve. However, the present disclosure is not meant to be limited to such and other types of valves are contemplated.

The controller 62 of the injection device 10′ is in signal communication with the first and second drive mechanisms 26, 54 in addition to the third drive mechanism 70. As a result, the controller 62 can selectively and individually direct the first, second, and third drive mechanisms 26, 54, 70 to force the first drug, gas, and second drug from the first, second, and third chambers 22a, 50a, 66a, respectively, to the output 38 in a predetermined sequence. The predetermined sequence can be stored in a memory of the controller 62, manually input by a user, or can be provided to the controller 62 wirelessly from a remote device. Similarly, the controller 62 is in signal communication with the first, second, and third valves 46, 58, 74 via signal connections 64b, 64d, 64f, respectively. As a result, the controller 62 can selectively and individually control actuation of each of the first, second, and third valves 46, 58, 74. One embodiment of the predetermined sequence can comprise a discrete amount of the gas followed by a discrete amount of the first drug, which is then followed by a discrete amount of the second drug. However, potential embodiments of the predetermined sequences that the injection device 10′ is capable of injecting are described further below.

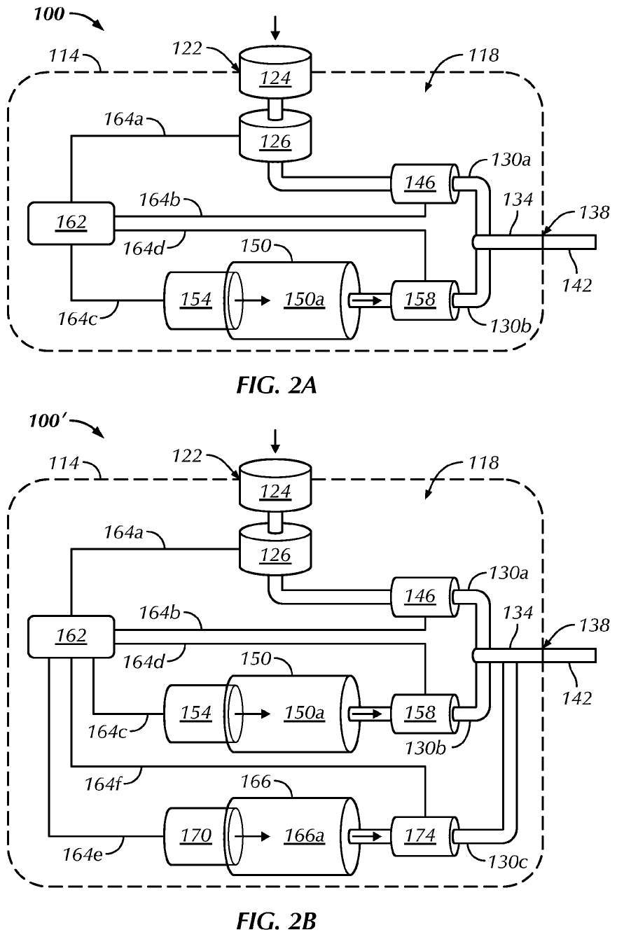

Now referring to FIG. 2A, another embodiment of the present disclosure comprises an injection device 100. The injection device 100 can have a housing 114 that is substantially hollow and is comprised of a metal or polymer. In one embodiment, the housing 114 can have an adhesive directly or indirectly attached thereto such that the injection device 100 is a wearable device. However, it is contemplated that the injection device 100 can comprise an injection device other than a wearable device. For example, the injection device can comprise an autoinjector, an infusion pump, or an injector configured to deliver a substance to a patient through a catheter. However, other devices capable of injecting a substance are contemplated.

The housing 114 can define a cavity 118 therein, where components of the injection device 100 are configured to reside within the cavity 118. The housing 114 also can define an input 122 and an output 138, which will each be described further below. Unlike the injection devices 10, 10′, the injection device 100 can receive gas directly from the atmosphere through the input 122. To selectively draw the gas from the input 122 and force the gas to the output 138, the injection device 100 can include a first drive mechanism 126 in fluid communication with the input 122. In one embodiment, the first drive mechanism 126 can comprise an electromechanical pump, though other types of pumps are contemplated for drawing the gas through the input 122 and forcing the gas to the output 138. The injection device 100 can also include a filter 124 in fluid communication with the input 122, where the filter 124 is configured to separate any unwanted particulates from the gas entering the injection device 100 through the input 122. Specifically, the filter 124 can be positioned at the input 122, within the first drive mechanism 126, or fluidly between the input 122 and the first drive mechanism 126. As a result, the filter 124 can function to prevent contaminants from entering the patient with the gas.

As stated above, the first drive mechanism 126 can be selectively actuated so as to draw the gas through the input 122 and force the gas to the output 138. To accomplish this, the first drive mechanism 126 can be in signal communication with a controller 162 through a signal connection 164a. The signal connection 164a can comprise a wired and/or a wireless connection. The controller 162 can comprise any suitable computing device configured to provide a software application for monitoring and controlling various operations of the injection device as described herein. For example, the controller 162 can include a processor, a memory, and/or a human-machine interface (HMI) device. The memory can be volatile (e.g., some types of RAM, etc.), non-volatile (e.g., ROM, flash memory, etc.), or a combination thereof. Further, the controller 162 can include additional storage devices, such as removable storage devices and/or non-removable storage devices. Exemplary HMI devices can comprise buttons, soft keys, speakers, display screens, lights, microphones, etc. As such, the controller 162 can be directly controlled by a user of the injection device 100. Alternatively, the controller 162 can include automated operation instructions such that the controller 162 can automatically control the injection device according to a predetermined operation automatically or upon instruction from the user. Further, the controller 162 can be wirelessly controlled by a remote device (not shown), such as through Bluetooth, WIFI, near-field communication (NFC), etc., as well as be configured to wirelessly transmit and/or receive operational information to and/or from such a remote device.

Continuing with FIG. 2A, the first drive mechanism 126 is configured to selectively draw the gas from the input 122 and force the gas to the output 138. The injection device 100 can include a first fluid path 130a that extends from the first drive mechanism 126 to an outlet path 134. The first fluid path 130a and the outlet path 134 can comprise any conventional fluid path capable of receiving and directing a flow of a gas. For example, the first fluid path 130a and/or the outlet path 134 can be a rigid or flexible tube comprised of metal, polymer, rubber, etc. The outlet path 134 can extend from the first fluid path 130a to an output 138 defined by the housing 114 of the injection device 100. At the output 138, the outlet path 134 can be in fluid communication with a cannula 142 extending from and/or at least partially exterior to the housing 114. Though depicted as exterior to and extending from the housing 114, it is contemplated that the cannula 142 is movable relative to the housing 114. For example, the cannula 142 can be moveable from a retracted position in which the cannula 142 is positioned at least partially within the housing 114 to an extended position in which the cannula 142 at least partially extends from the housing 114. In one embodiment, the cannula 142 comprises a hollow needle with a sharp tip for piercing the skin of a user and subcutaneously delivering a substance, where the needle can be linear or have angularly extending portions. However, other cannula embodiments are contemplated. Additionally, it is contemplated that the cannula 142 can be spaced a distance from the housing 114, such as when the injection device 100 comprises an infusion pump.

To further control the flow of the gas from the input 122 to the outlet path 134, a first valve 146 can be fluidly positioned between the first drive mechanism 126 and the output 138. Specifically, the first valve 146 can be incorporated into the first fluid path 130a. In operation, the first valve 146 is configured to selectively inhibit the flow of the gas from the first drive mechanism 126 to the output 138. To accomplish this, the first valve 146 can be in signal communication with the controller 162 through signal connection 164b, which can be a wired and/or wireless connection. The controller 162 can thus instruct the first valve 146 to selectively block or allow flow of the drug to the output 138 based upon a predetermined dispensing sequence, as will be described further below. The first valve 46 can be a solenoid valve, electrostatic valve, piezoelectric valve, shape-memory actuated valve, coaxial valve, or an angle seat valve. However, the present disclosure is not meant to be limited to such and other types of valves are contemplated.

With continued reference to FIG. 2A, the injection device 100 can further include a first reservoir 150 disposed within the internal cavity 118, where the first reservoir 150 defines a first chamber 150a. The first reservoir 150 can be configured as a polymer or glass syringe or cartridge configured to be inserted into the injection device 100 upon preparation or assembly of the injection device. Such a reservoir can be removably disposed within the cavity 118, such that a user can remove the first reservoir 150 upon depletion of its contents and replace the first reservoir 150. However, it is contemplated that the first reservoir 150 may be irremovably disposed within the cavity 118. In another embodiment, it is contemplated that the first reservoir 150 can define a receiving space incorporated into the design of the injection device 100 and is configured to be filled from an external source.

The first chamber 150a of the first reservoir 150 is configured to store a drug. The drug, like the gas, can be introduced to a patient through the cannula 142. Additionally, the first chamber 150a is in fluid communication with the output 138. To force the drug from the first chamber 150a to the output 138, the injection device 100 can include a second drive mechanism 154 operably attached to the first reservoir 150. The second drive mechanism 154 can be configured to selectively force the drug from the first chamber 150a, as will be described below. In one embodiment, the second drive mechanism 154 can comprise a motor operably connected to a TSA configured to axially advance a plunger (not shown) through the first chamber 150a so as to force the drug from the first reservoir 150. However, it is also contemplated that the second drive mechanism 154 can include an electromechanical pump, piezoelectric actuator, shape memory alloy, peristaltic pump, spring, electro-chemical drive mechanism, and/or any other suitable device for causing axial actuation of a plunger.

As stated above, the second drive mechanism 154 can be selectively actuated so as to force the drug from the first reservoir 150. To accomplish this, the second drive mechanism 154 can be in signal communication with a controller 162 through a signal connection 164c. The signal connection 164c can comprise a wired and/or a wireless connection. The injection device 100 can include a second fluid path 130b that extends from the first chamber 150a to the outlet path 134. The second fluid path 130b, like the first fluid path 130a and the outlet path 134, can comprise any conventional fluid path capable of receiving and directing a flow of a drug. For example, the second fluid path 130b can be a rigid or flexible tube comprised of metal, polymer, rubber, etc.

To further control the flow of the drug from the first chamber 150a to the outlet path 134, a second valve 158 can be fluidly positioned between the first chamber 150a and the output 138. Specifically, the second valve 158 can be incorporated into the second fluid path 130b. In operation, the second valve 158 is configured to selectively inhibit the flow of the drug from the first chamber 150a to the output 138. To accomplish this, the second valve 158 can be in signal communication with the controller 162 through signal connection 164d, which can be a wired and/or wireless connection. The controller 162 can thus instruct the second valve 158 to selectively block or allow flow of the drug to the output 138 based upon a predetermined dispensing sequence, as will be described further below. The second valve 158 can be a solenoid valve, electrostatic valve, piezoelectric valve, shape-memory actuated valve, coaxial valve, or an angle seat valve. However, the present disclosure is not meant to be limited to such and other types of valves are contemplated.

As described above, the controller 162 is in signal communication with the first and second drive mechanisms 126, 154 via signal connections 164a, 164c, respectively. As a result, the controller 162 can selectively and individually direct the first and second drive mechanisms 126, 154 to force the gas and drug from the input and first chamber 122, 150a, respectively, to the output 38 in a predetermined sequence. The predetermined sequence can be stored in a memory of the controller 162, manually input by a user, or can be provided to the controller 162 wirelessly from a remote device. Similarly, the controller 162 is in signal communication with the first and second valves 146, 158 via signal connections 164b, 164d, respectively. As a result, the controller 162 can selectively and individually control actuation of each of the first and second valves 146, 158. One embodiment of the predetermined sequence can comprise a discrete amount of the gas followed by a discrete amount of the drug. However, potential embodiments of the predetermined sequences that the injection device 100 is capable of injecting are described further below.

Now referring to FIG. 2B, another embodiment of an injection device 100′ is shown. The injection device 100′ has similar components to the injection device 100. As a result, like reference numbers will be utilized in connection with the injection device 100′, and descriptions of similar components will not be repeated for brevity. As shown in FIG. 2B, the injection device 100′ can include a second reservoir 166 disposed within the internal cavity 118, where the second reservoir 166 defines a second chamber 166a. Like the first reservoir 150, the second reservoir 166 can be configured as a polymer or glass syringe or cartridge configured to be inserted into the injection device 100′ upon preparation or assembly of the injection device. Such a reservoir can be removably disposed within the cavity 118, such that a user can remove the second reservoir 166 upon depletion of its contents and replace the second reservoir 166. However, it is contemplated that the second reservoir 166 may be irremovably disposed within the cavity 118. In another embodiment, it is contemplated that the second reservoir 166 can define a receiving space incorporated into the design of the injection device 100 and is configured to be filled from an external source.

The second chamber 166a of the second reservoir 166 is configured to store a second drug or liquid substance (buffer, excipient, etc.) that can enhance the effect or comfort of the first drug and/or injection of gas, where the drug stored by the first reservoir 150 can be considered a first drug. It is contemplated that the first and second drugs can be the same, such that the second reservoir 166 represents an additional supply of the same drug contained within the first reservoir 150. However, it is further contemplated that the first and second drugs can be different. For example, the first and second drugs can comprise drugs that are injected into a patient during the same injection process but cannot be mixed prior to injection or the efficacy of a therapy is improved via injection of a second drug or liquid substance. The second chamber 166a is in fluid communication with the output 138. To force the second drug from the second chamber 166a to the output 138, the injection device 100′ can include a third drive mechanism 170 operably attached to the second reservoir 166. The third drive mechanism 170 can be configured to selectively force the second drug from the second chamber 166a as will be described below. In one embodiment, the third drive mechanism 170 can comprise a motor operably connected to a TSA configured to axially advance a plunger (not shown) through the second chamber 166a so as to force the second drug from the second reservoir 166. However, it is also contemplated that the third drive mechanism 170 can include an electromechanical pump, piezoelectric actuator, shape memory alloy, peristaltic pump, spring, electro-chemical drive mechanism, and/or any other suitable device for causing axial actuation of a plunger.

As stated above, the third drive mechanism 170 can be selectively actuated so as to force the second drug from the second reservoir 166. To accomplish this, the third drive mechanism 170 can be in signal communication with the controller 162 through a signal connection 164e. The signal connection 164e can comprise a wired and/or a wireless connection. The third drive mechanism 170 is configured to selectively force the drug from the second chamber 166a. The injection device 100′ can include a third fluid path 130c that extends from the second chamber 166a to the outlet path 134. The third fluid path 130c, like the first fluid path 130a, second fluid path 130b and the outlet path 134, can comprise any conventional fluid path capable of receiving and directing a flow of a drug. For example, the third fluid path 130c can be a rigid or flexible tube comprised of metal, polymer, rubber, etc. The configuration of first, second, and third fluid paths 130a-130c shown in FIG. 1B is illustrative and different combinations and embodiments of fluid communication are envisaged

To further control the flow of the drug from the second chamber 166a to the outlet path 134, a third valve 174 can be fluidly positioned between the second chamber 166a and the output 138. Specifically, the third valve 174 can be incorporated into the third fluid path 130c. In operation, the third valve 174 is configured to selectively inhibit the flow of the drug from the third chamber 166a to the output 38. To accomplish this, the third valve 174 can be in signal communication with the controller 162 through signal connection 164f, which can be a wired and/or wireless connection. The controller 162 can thus instruct the third valve 174 to selectively block or allow flow of the gas to the output 138 based upon a predetermined dispensing sequence, as will be described further below. The third valve 174 can be a solenoid valve, electrostatic valve, piezoelectric valve, shape-memory actuated valve, coaxial valve, or an angle seat valve. However, the present disclosure is not meant to be limited to such and other types of valves are contemplated.

The controller 162 of the injection device 100′ is in signal communication with the first and second drive mechanisms 126, 154 in addition to the third drive mechanism 170. As a result, the controller 162 can selectively and individually direct the first, second, and third drive mechanisms 126, 154, 170 to force the gas, first drug, and second drug from the input 122 and the first and second chambers 146a, 166a, respectively, to the output 138 in a predetermined sequence. The predetermined sequence can be stored in a memory of the controller 162, manually input by a user, or can be provided to the controller 162 wirelessly from a remote device. Similarly, the controller 162 is in signal communication with the first, second, and third valves 146, 158, 174 via signal connections 164b, 164d, 164f, respectively. As a result, the controller 162 can selectively and individually control actuation of each of the first, second, and third valves 146, 168, 174. One embodiment of the predetermined sequence can comprise a discrete amount of the gas followed by a discrete amount of the first drug, which is then followed by a discrete amount of the second drug or liquid substance. However, potential embodiments of the predetermined sequences that the injection device 100′ is capable of injecting are described further below.

Now referring to FIGS. 3A-3D, process flow diagrams of methods of injecting a drug and gas using the injection device 10, 100 according to various predetermined sequences are shown. As shown in FIG. 3A, the injection device 10, 100 can inject the drug before the gas, and repeat this sequence n cycles as needed. As shown in FIG. 3B, the injection device 10, 100 can inject the gas before the drug, and repeat this sequence n cycles as needed. FIG. 3C shows a sequence in which an amount of the gas is injected by the injection device 10, 100, followed by an amount of the drug, followed by another amount of the gas. This process can also by repeated n cycles. Similarly, FIG. 3D shows a sequence in which an amount of the drug is injected by the injection device 10, 100, followed by an amount of the gas, followed by another amount of the drug. Similarly, this process can be repeated n cycles as needed. Though a particular number of predetermined sequences for injecting the drug and gas using the injection device 10, 100 are shown and described with reference to FIGS. 3A-3D, the present disclosure is not intended to be limited to such. The sequences may either be performed consecutively or after a pre-determined period of time. Additionally, the volumes and/or ratios of the drug and gas inject may differ between respective sequences. Further, for each process n cycles can comprise any number of cycles as needed/desired.

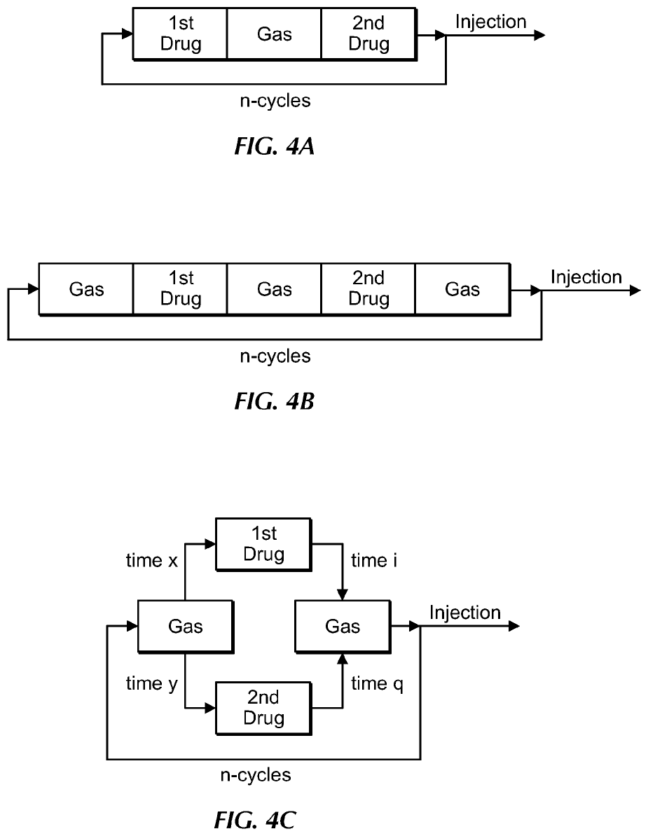

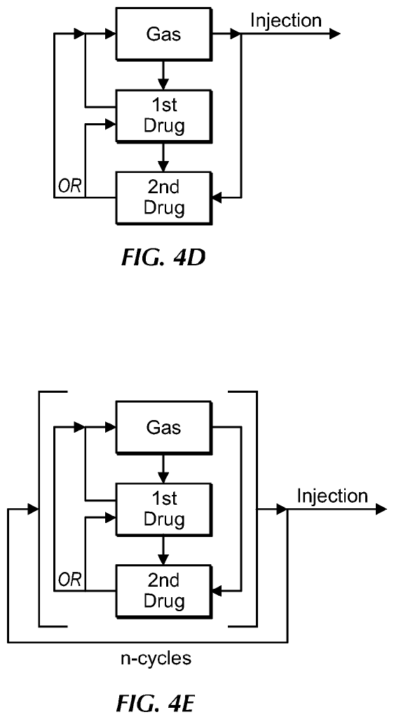

Referring to FIGS. 4A-4E, process flow diagrams of methods of injecting a first drug, a second drug, and a gas using the injection device 10′, 100′ according to various predetermined sequences are shown. As shown in FIG. 4A, the injection device 10′, 100′ can inject the first drug before the gas, which is then followed by the second drug. This sequence can be repeated n cycles as needed. As shown in FIG. 4B, the injection device 10′, 100′ can inject the gas first, followed by the first drug, the gas, the second drug, and then the gas again, the sequence of which can be repeated as needed. Likewise, this process can be repeated n cycles as needed. In FIG. 4C, the injection device 10′, 100′ can inject the gas first, followed by the first and second drugs simultaneously, followed by another amount of the gas, which can be repeated n cycles as desired. FIG. 4D shows that the injection device 10′, 100′ can be programmed with different time intervals between dosing of the gas and the first and/or second drugs. FIG. 4E shows that the injection device 10′, 100′ can be programmed to repeat certain orders in delivery cycles.

While the invention is described herein using a limited number of embodiments, these specific embodiments are not intended to limit the scope of the invention as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in any particular order as desired.

What is claimed:

1. An injection device for sequentially and parenterally providing a drug and a gas to a patient, the injection device comprising:
a housing defining an internal cavity and an output;
a first reservoir disposed within the internal cavity, wherein the first reservoir defines a first chamber that is configured to store a drug and is in fluid communication with the output;
a first drive mechanism operably attached to the first reservoir so as to selectively force the drug from the first chamber;
a second reservoir disposed within the internal cavity, wherein the second reservoir defines a second chamber that is configured to store a gas and is in fluid communication with the output;
a second drive mechanism operably attached to the second reservoir so as to selectively force the gas from the second chamber; and
a controller in signal communication with the first and second drive mechanisms, such that the controller is configured to selectively and independently direct the first and second drive mechanisms to force the drug and gas from the first and second chambers, respectively, to the output in a predetermined sequence.

2. The injection device of claim 1, further comprising:
a first valve fluidly positioned between the first chamber and the output, wherein the first valve is configured to selectively inhibit flow of the drug from the first chamber to the output.

3. The injection device of claim 2, further comprising:
a second valve fluidly positioned between the second chamber and the output, wherein the second valve is configured to selectively inhibit flow of the gas from the second chamber to the output.

4. The injection device of claim 3, wherein the first and second valves are solenoid valves, electrostatic valves, piezoelectric valves, shape-memory actuated valves, coaxial valves, or angle seat valves.

5. The injection device of claim 3, wherein the controller is in signal communication with the first and second valves so as to selectively and individually control actuation of each of the first and second valves.

6. The injection device of claim 1, wherein the drug is a first drug, the injection device further comprising:
a third reservoir disposed within the internal cavity, wherein the third reservoir defines a third chamber that is configured to store a second drug and is in fluid communication with the output; and
a third drive mechanism operably attached to the third reservoir so as to selectively force the second drug from the third chamber,
wherein the controller is in signal communication with the third drive mechanism such that the controller is configured to selectively direct the third drive mechanism to force the second drug from the third chamber as part of the predetermined sequence.

7. The injection device of claim 6, further comprising:
a third valve fluidly positioned between the third chamber and the output, wherein the third valve is configured to selectively inhibit flow of the second drug from the third chamber to the output.

8. The injection device of claim 7, wherein the controller is in signal communication with the third valve so as to selectively control actuation of the third valve.

9. The injection device of claim 1, wherein the first and second drive mechanisms include electromechanical pumps, telescoping screw assemblies, piezoelectric actuators, shape memory alloy, peristaltic pumps, springs, or electro-chemical drive mechanisms.

10. The injection device of claim 1, further comprising:
a cannula extending from the output and configured to direct the drug and the gas to a patient.

11. An injection device for sequentially and parenterally providing a drug and a gas to a patient, the injection device comprising:
a housing defining an internal cavity, an input, and an output;
a first drive mechanism in fluid communication with the input, where the first drive mechanism is configured to selectively draw gas through the input and force the gas to the output;
a first reservoir disposed within the internal cavity, wherein the first reservoir defines a first chamber that is configured to store a drug and is in fluid communication with the output;
a second drive mechanism operably attached to the first reservoir so as to selectively force the drug from the first chamber; and
a controller in signal communication with the first and second drive mechanisms, such that the controller is configured to selectively and independently direct the first and second drive mechanisms to force the gas and drug from the input and first chamber, respectively, to the output in a predetermined sequence.

12. The injection device of claim 11, further comprising:
a first valve fluidly positioned between the input and the output, wherein the first valve is configured to selectively inhibit flow of the gas from the input to the output.

13. The injection device of claim 12, further comprising:
a second valve fluidly positioned between the first chamber and the output, wherein the second valve is configured to selectively inhibit flow of the drug from the first chamber to the output.

14. The injection device of claim 13, wherein the first and second valves are solenoid valves, electrostatic valves, piezoelectric valves, shape-memory actuated valves, coaxial valves, or angle seat valves.

15. The injection device of claim 13, wherein the controller is in signal communication with the first and second valves so as to selectively and individually control actuation of each of the first and second valves.

16. The injection device of claim 11, wherein the drug is a first drug, the injection device further comprising:
a second reservoir disposed within the internal cavity, wherein the second reservoir defines a second chamber that is configured to store a second drug and is in fluid communication with the output; and
a third drive mechanism operably attached to the second reservoir so as to selectively force the second drug from the second chamber,
wherein the controller is in signal communication with the third drive mechanism such that the controller is configured to selectively direct the third drive mechanism to force the second drug from the second chamber as part of the predetermined sequence.

17. The injection device of claim 16, further comprising:
a third valve fluidly positioned between the second chamber and the output, wherein the third valve is configured to selectively inhibit flow of the second drug from the second chamber to the output.

18. The injection device of claim 17, wherein the controller is in signal communication with the third valve so as to selectively control actuation of the third valve.

19. The injection device of claim 11, wherein the first and second drive mechanisms include electromechanical pumps, telescoping screw assemblies, piezoelectric actuators, shape memory alloy, peristaltic pumps, springs, or electro-chemical drive mechanisms.

20. The injection device of claim 11, further comprising:
a filter in fluid communication with the input.
